(19) United States
(12) Patent Application Publication
(10) Pub. No.: US 2026/0165648 A1
(43) Pub. Date: Jun. 18, 2026

(54) MEASUREMENT DEVICE AND RECORDING MEDIUM

(71) Applicant: HIOKI E.E. CORPORATION, Nagano (JP)

(72) Inventors: Koki NAKAZAWA, Nagano (JP); Hiroki SAITO, Nagano (JP); Koichi TARASAWA, Nagano (JP); Kenta IKEDA, Nagano (JP); Yuki KARASAWA, Nagano (JP); Peiyu XIA, Nagano (JP); Yukiya SAITO, Nagano (JP)

(21) Appl. No.: 18/718,284

(22) PCT Filed: Nov. 30, 2022
(86) PCT No.: PCT/JP2022/044287

§ 371 Date: Jun. 10, 2024

(30) Foreign Application Priority Data
Dec. 15, 2021 (JP) 2021-203454

(51) Int. Cl. A61B 5/00; A61B 5/05

(52) U.S. Cl. CPC A61B 5/6801; A61B 5/0006; A61B 5/0008; A61B 5/05; A61B 5/681; A61B 5/7257; A61B 5/746

(57) ABSTRACT

A measuring device that communicates with a sensor acquires, from the sensor, information on a detection amount specific to the sensor and the detection amount detected by the sensor. Then, the measuring device corrects the acquired detection amount based on the information related to the detection amount specific to sensor. Then, the measuring device calculates the measurement amount of the measurement target based on the corrected detection amount.

### TECHNICAL FIELD

The present invention relates to a measuring device and a recording medium.

### BACKGROUND ART

JP2018-13499A discloses a measuring device that measures a measurement amount based on a detection amount detected by a sensor.

### SUMMARY OF INVENTION

In the above-described measuring device, an output terminal of a sensor for detecting the measurement amount of a measurement target is electrically connected to an input terminal of the measuring device. Then, the measuring device acquires, from the sensor, the detection amount of the sensor, and calculates the measurement amount of the measurement target based on the detection amount of the sensor.

However, the detection amount of the sensor acquired by the measuring device includes an error caused by a detection characteristic of the sensor or a detection performance such as an individual difference of the sensor itself. Therefore, there is a problem in that the measurement amount calculated based on the detection amount of the sensor also includes an error component caused by the detection performance of the sensor.

The present invention has been made in view of such a problem, and an object of the present invention is to acquire a measurement amount in consideration of the detection performance of a sensor.

According to an aspect of the present invention, a measuring device for communicating with a sensor includes: an acquirer configured to acquire, from the sensor, information on a detection amount specific to the sensor and the detection amount detected by the sensor; a processor configured to correct the detection amount acquired by the acquirer based on the information; and a calculator configured to calculate a measurement amount of a measurement target based on the detection amount corrected by the processor.

According to the present aspect, when the measurement amount is calculated, an error caused by the detection performance of the sensor can be considered by using the information regarding the detection amount specific to the sensor acquired from the sensor itself. Therefore, the measurement amount in consideration of the detection performance of the sensor can be acquired.

### BRIEF DESCRIPTION OF DRAWINGS

FIG. 1 is a diagram illustrating a configuration of a measuring device according to an embodiment of the present invention.

FIG. 2 is a conceptual diagram illustrating an example of a format of sensor information stored in a current sensor.

FIG. 3 is a conceptual diagram illustrating an example of a format of sensor information stored in a voltage sensor.

FIG. 4 is a block diagram illustrating a functional configuration of the measuring device in the present embodiment.

FIG. 5 is a conceptual diagram illustrating an example of phase characteristic data included in the sensor information.

FIG. 6 is a flowchart illustrating a processing procedure example of a measurement method executed by the measuring device.

### DESCRIPTION OF EMBODIMENTS

Hereinafter, embodiments of the present invention will be described with reference to the accompanying drawings.

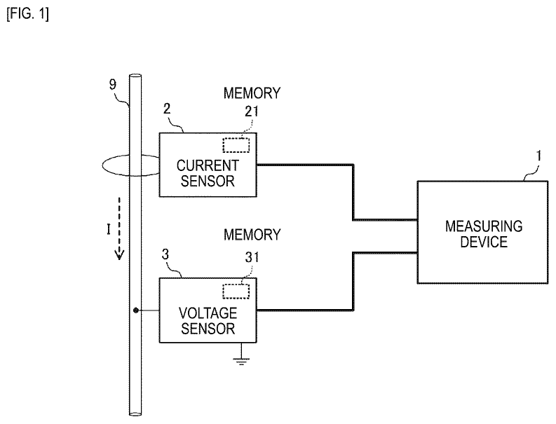

FIG. 1 is a block diagram illustrating a configuration of a measuring device connected to a plurality of sensors according to an embodiment of the present invention.

A measuring device 1 is a measuring device that uses one or more sensors to analyze a state of a measurement target 9. The measuring device 1 is realized by, for example, a power analyzer, a voltage meter, a current meter, a magnetic field meter, or an optical meter.

Examples of the measurement target 9 include an electric line for transmitting electric power, an object having electric resistance, light, and a magnetic flux density. The measurement target 9 in the present embodiment is an electric line for transmitting an AC power. Hereinafter, a frequency of an AC current flowing through the electric line is referred to as a measurement frequency.

In the present embodiment, the measuring device 1 outputs the AC power of the measurement target 9 based on the output signals of a current sensor 2 and a voltage sensor 3. The measuring device 1 is a computer including a processor, a read only memory (ROM), a random access memory (RAM), an input/output interface, and a bus connecting these components to each other.

Each of the current sensor 2 and the voltage sensor 3 is a sensor that detects a detection amount of the measurement target 9, and stores (holds) information on the detection amount specific to the sensor in an internal ROM or RAM. This sensor performs communication between the sensor itself and the measuring device 1, and is connected to the measuring device 1 in a wired or wireless manner.

Hereinafter, information on the detection amount specific to the sensor is referred to as “sensor information”. The sensor information mentioned here includes at least one of specific information determined for each type of sensor and specific information determined for each individual sensor of the same type.

The current sensor 2 detects a detection amount related to a current I flowing through the measurement target 9. For example, the current sensor 2 includes a magnetic sensor that detects a flux generated by the current I of the measurement target 9, and detects a voltage acquired by detecting the flux using the magnetic sensor as the detection amount related to the current. The current sensor 2 performs communication between the sensor itself and the measuring device 1, converts a time-series detection amount related to the magnitude of the current I into a detection signal, and outputs the detection signal to the measuring device 1 in a wired or wireless manner. The current sensor 2 includes, for example, a coil formed by winding a wire around a magnetic core such as an iron core. The current sensor 2 may be of a penetrating type or a clamp type.

The current sensor 2 includes a memory 21. The memory 21 is a non-volatile memory and is realized by, for example, an electrically erasable programmable read-only memory (EEPROM). The sensor information of the current sensor 2 itself is stored in the memory 21.

The sensor information of the current sensor 2 includes, for example, sensor identification data for identifying the current sensor 2, detection characteristic data indicating the frequency characteristic of the phase and amplitude of the current sensor 2, and the like.

The voltage sensor 3 detects a detection amount related to the magnitude of the voltage V generated in the measurement target 9. For example, the voltage sensor 3 includes an electrode for detecting the voltage of the measurement target 9 in a non-contact manner and a circuit for applying a voltage to the electrode. The voltage sensor 3 detects, as a detection amount related to the voltage, a voltage acquired by generating a voltage in the electrode so that a current does not flow between the electrode and the measurement target 9. The voltage sensor 3 performs communication between the sensor itself and the measuring device 1, converts the detected time-series detection amount into a detection signal, and outputs the detection signal to the measuring device 1 in a wired or wireless manner.

The voltage sensor 3 includes a memory 31 similarly to the configuration of the current sensor 2. The memory 31 is a non-volatile memory and is realized by, for example, an EEPROM. The memory 31 holds sensor information of the voltage sensor 3 itself. The sensor information includes sensor identification data, detection characteristic data, and the like.

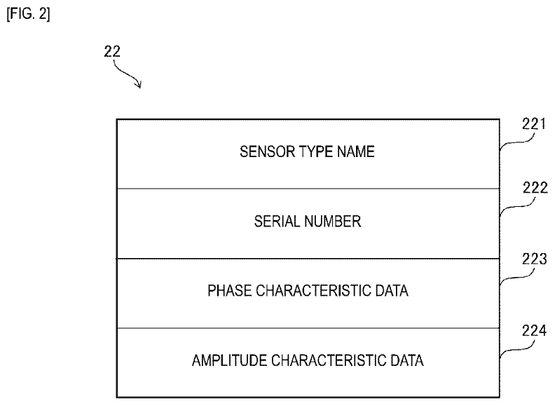

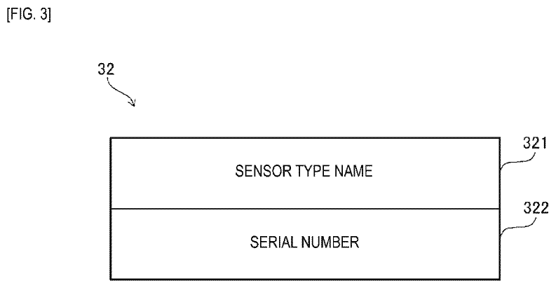

Next, sensor information stored in each of the current sensor 2 and the voltage sensor 3 will be described with reference to FIGS. 2 and 3.

FIG. 2 is a conceptual diagram illustrating an example of sensor information 22 stored in the memory 21 of the current sensor 2.

As illustrated in FIG. 2, the sensor information 22 includes a sensor type name 221, a serial number 222, phase characteristic data 223, and amplitude characteristic data 224.

The sensor type name 221 and the serial number 222 are sensor identification data for identifying the current sensor 2.

The sensor type name 221 is an identifier for identifying the performance and structure of the current sensor 2, and the serial number 222 is an identification number specific to the current sensor 2.

The phase characteristic data 223, the amplitude characteristic data 224, and the rated current 225 are detection characteristic data indicating the current detection characteristic of the current sensor 2.

The phase characteristic data 223 is information indicating a frequency characteristic of a phase related to a detection amount detected by the current sensor 2. For example, the phase characteristic data 223 is formed by a correspondence table indicating, for each specific frequency, a phase delay of the detection amount caused by the current sensor 2 with respect to the AC voltage.

Similarly, the amplitude characteristic data 224 is information indicating the frequency characteristic of the amplitude related to the detection amount detected by the current sensor 2. For example, the amplitude characteristic data 224 is formed by a correspondence table indicating, for each specific frequency, a phase delay of the detection amount caused by the voltage sensor 3 with respect to the AC voltage.

The phase characteristic data 223 and the amplitude characteristic data 224 in the present embodiment are correspondence tables indicating the detection characteristic specific to the current sensor 2. The phase characteristic data 223 and the amplitude characteristic data 224 may be a correspondence table indicating the standard detection characteristic of the current sensor 2, or may be function data indicating a function defining the detection characteristics.

As described above, the sensor identification data and the detection characteristic data of the current sensor 2 are stored as the sensor information 22 in the memory 21 of the current sensor 2.

FIG. 3 is a conceptual diagram illustrating an example of sensor information 32 stored in the memory 31 of the voltage sensor 3.

As illustrated in FIG. 3, the sensor information 32 includes a sensor type name 321 and a serial number 322.

The sensor type name 321 and the serial number 322 correspond to the sensor type name 221 and the serial number 222 included in the sensor information 22 described above, respectively.

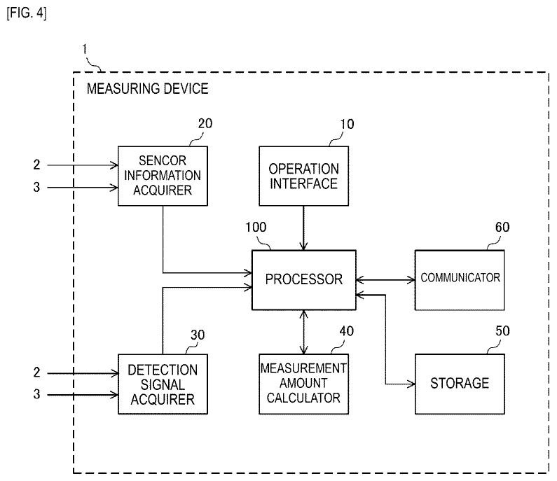

FIG. 4 is a block diagram illustrating a functional configuration of the measuring device 1 according to the present embodiment.

The measuring device 1 includes an operation interface 10, a sensor information acquirer 20 corresponding to a first acquirer, a detection signal acquirer 30 corresponding to a second acquirer, a measurement amount calculator 40, a storage 50, a communicator 60, and a processor 100.

The operation interface 10 includes a plurality of push buttons provided around a display screen, a touch sensor disposed in the display screen, a keyboard, and a mouse, or the like. The operation interface 10 receives an input operation of a person who uses the measuring device 1, and generates an operation signal indicating the content of the received input operation.

Examples of the input operation include an operation of pressing a power button, an operation of setting a measurement condition, an operation of instructing execution of measurement processing for measurement of AC power of the measurement target 9, and an operation of instructing termination of the measurement processing.

When the operation interface 10 receives an input operation for setting a measurement condition by the user, the operation interface 10 outputs an operation signal indicating the measurement condition to the processor 100 in order to record the operation signal in the storage 50. In addition, when the operation interface 10 receives an input operation of instructing the execution of the measurement process by the user, the operation interface 10 outputs an operation signal indicating the content of the input operation to the processor 100.

The sensor information acquirer 20 functions as an acquirer that acquires sensor information from a sensor connected to the measuring device 1.

The sensor information acquirer 20 in the present embodiment communicates with each of the current sensor 2 and the voltage sensor 3 to acquire the sensor information 22 and the sensor information 32 from each of the sensors. For example, the sensor information acquirer 20 acquires the detection characteristic data included in the sensor information 22 from the current sensor 2. The sensor information acquirer 20 outputs the acquired sensor information 22 and 32 of each sensor to the processor 100.

The detection signal acquirer 30 functions as an acquirer for acquiring, from a sensor connected to the measuring device 1, a detection amount detected by the sensor.

The detection signal acquirer 30 acquires, from each of the current sensor 2 and the voltage sensor 3, a detection signal acquired by converting a detection amount detected by each sensor. The detection signal acquirer 30 outputs the detection amount extracted from the acquired detection signal of each sensor to the processor 100.

The measurement amount calculator 40 functions as a calculator that calculates the measurement amount of the measurement target 9 based on the detection amount acquired by the processor 100. Examples of the measurement amount to be calculated include a physical quantity indicating a quantity such as a current, a potential, electric power, or a magnetic field, and a chemical quantity indicating a quantity such as that of a chemical substance.

In the present embodiment, the measurement amount calculator 40 calculates the effective value of the current I flowing through the measurement target 9 based on the detection amount of the current sensor 2 output from the processor 100. Further, the measurement amount calculator 40 calculates the effective value of the voltage V generated in the measurement target 9 based on the detection amount extracted from the detection signal output from the voltage sensor 3. Further, the measurement amount calculator 40 calculates a phase difference of the current I with respect to the voltage V generated in the measurement target 9.

Then, the measurement amount calculator 40 calculates the average value of the power P in the measurement target 9 using the calculated effective value Iᵣₘₛ of the current I, the calculated effective value Vᵣₘₛ of the voltage V, and the phase difference θ as in the following Equation (1).

$$P = {I}_{rms} * {V}_{rms} \times cos θ \qquad ( 1 )$$

In this manner, the measurement amount calculator 40 calculates the measurement amount indicating the magnitude of the power P transmitted to the measurement target 9 based on the detection amount acquired by each sensor. The measurement amount calculator 40 outputs the calculated measurement amount to the processor 100 as a measurement result.

The storage 50 stores a measurement condition and a measurement result. For example, as the measurement result, time-series data indicating the values of the current I and the voltage V calculated by the measurement amount calculator 40 in time-series and the average value of the power P are stored in the storage 50. A part of the storage 50 may be configured by, for example, an external memory such as a USB memory connectable to the measuring device 1.

In addition, the storage 50 stores a program for the processor 100 to execute the measurement process according to the present embodiment. That is, the storage 50 is a computer-readable recording medium in which a program for controlling each unit of the measuring device 1 is recorded.

The communicator 60 performs communication between the measuring device 1 and an external device. The communicator 60 transmits or receives a measurement condition or a measurement result to or from an external device through a network such as the Internet or a telephone network. The communicator 60 is realized by, for example, a communication circuit.

The processor 100 functions as a processor that corrects the detection amount acquired by the detection signal acquirer 30 based on the sensor information 22 and the sensor information 32 acquired by the sensor information acquirer 20.

The processor 100 is a processor that controls each unit of the measuring device 1. Examples of the processor include a central processing unit (CPU) and a micro processor unit (MPU).

In the present embodiment, the processor 100 acquires the sensor information 22 of the current sensor 2 and the sensor information 32 of the voltage sensor 3 from the sensor information acquirer 20, and records the sensor information 22 and the sensor information 32 in the storage 50.

Then, when the processor 100 acquires the detection amount of the current sensor 2 from the detection signal acquirer 30, the processor 100 corrects the acquired detection amount of the current sensor 2 based on the phase characteristic data 223 indicated in the sensor information 22 of the current sensor 2. Further, the processor 100 may correct the acquired detection amount of the current sensor 2 based on the amplitude characteristic data 224 indicated in the sensor information 22.

In a case where the voltage sensor 3 has a detection characteristic in which the frequency fluctuations of the phase and the amplitude are large, similarly to the current sensor 2, the phase characteristic data and the amplitude characteristic data of the voltage sensor 3 may be stored in the memory 31, and the processor 100 may correct the detection amount of the voltage sensor 3 based on the acquired data.

Thereafter, the processor 100 outputs the corrected detection amount of the current sensor 2 to the measurement amount calculator 40. Then, as described above, by using the corrected detection amount of the current sensor 2, the measurement amount calculator 40 calculates the measurement amounts such as the AC waveform of the current I, the effective value of the current I, and the average value of the power P. Thus, the accuracy of the measurement amount calculated by the measurement amount calculator 40 is improved.

In the example illustrated in FIG. 4, the sensor information and the detection amount of each sensor are acquired by the sensor information acquirer 20 and the detection signal acquirer 30 different from each other, but may be acquired by the same acquirer.

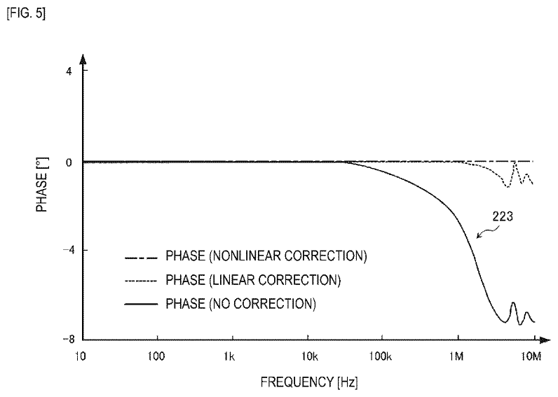

Next, a method of correcting the detection amount of the current sensor 2 based on the phase characteristic data indicated in the sensor information 22 of the current sensor 2 will be described with reference to FIG. 5.

FIG. 5 is a schematic diagram illustrating an example of the phase characteristic data 223 included in the sensor information 22 on the current sensor 2. Here, the vertical axis represents the phase of the detection amount of the current sensor 2 with respect to the AC voltage applied to the measurement target 9, and the horizontal axis represents the frequency of the detection amount of the current sensor 2.

The phase characteristic data 223 indicated by the solid line in FIG. 5 is formed by a plurality of frequency points indicating a phase delay defined for each frequency. In the high frequency band of the phase characteristic data 223, as the frequency of the AC current increases, the phase delay with respect to the AC voltage applied to the measurement target 9 becomes larger than the phase delay in the frequency band lower than the high frequency band.

On the other hand, it is also possible to linearly correct the detection amount of the current sensor 2 using the phase characteristic data indicating the phase delay of the frequency at one representative point within the detectable frequency range by the current sensor 2. In this case, the processor 100 acquires an approximate straight line indicating the relationship between the frequency and the phase delay of the AC current based on the phase delay of the frequency at one point, and calculates the phase delay corresponding to the measurement frequency in the approximate straight line. Then, the processor 100 converts the calculated phase delay into a delay time of the detection amount, and shifts the time-series detection amount acquired from the current sensor 2 by the time. In this manner, the processor 100 corrects the detection amount of the current sensor 2 as indicated by the broken line in FIG. 5.

However, in the method of calculating the phase delay of the measurement frequency by acquiring the approximate straight line, the detection amount may not be sufficiently corrected in the high frequency band as illustrated by the broken line in FIG. 5. As a countermeasure against this, non-linear correction is performed on the detection amount of the current sensor 2 using the phase characteristic data 223 in which the phase delay is defined at a plurality of frequency points.

Specifically, in order to accurately approximate the phase characteristic of the current sensor 2, the processor 100 acquires an approximate curve represented by a high-order equation by using the phase characteristic data 223 indicating the phase delays at a plurality of frequency points. Then, the processor 100 calculates a phase delay corresponding to the measurement frequency in the approximate curve, and applies the calculated phase delay to the detection amount of the current sensor 2, thereby correcting the detection amount of the current sensor 2 as indicated by the one dot chain line in FIG. 5.

Thus, even when the current I in the high-frequency band flowing through the measurement target 9 is detected using the current sensor 2, the detection amount of the current sensor 2 can be accurately corrected, and a decrease in measurement accuracy in the high frequency band due to the phase characteristic of the current sensor 2 can be suppressed.

In the example illustrated in FIG. 5, the processor 100 performs the linear correction or the non-linear correction on the detection amount of the current sensor 2 using the phase characteristic data 223, but similarly, the processor 100 can correct the detection amount of the current sensor 2 by acquiring an approximate straight line or an approximate curve using the amplitude characteristic data 224.

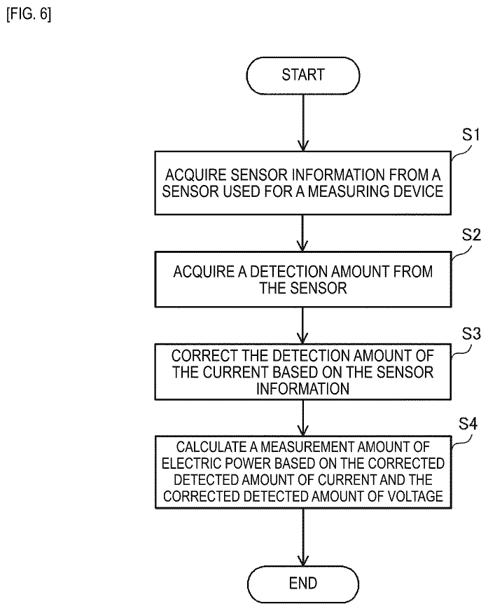

Next, the operation of the measuring device 1 in the present embodiment will be described with reference to FIG. 6.

FIG. 6 is a flowchart illustrating a processing procedure example of a measurement method for measure of the state of the measurement target 9.

In step S1, the measuring device 1 acquires, from the current sensor 2, the sensor information 22 including information on the detection amount specific to the current sensor 2. In addition, the measuring device 1 acquires, from the voltage sensor 3, sensor information 32 including information on the detection amount specific to the voltage sensor 3.

In step S2, the measuring device 1 acquires, from the current sensor 2, the detection amount related to the magnitude of the current I flowing through the measurement target 9. In addition, the measuring device 1 acquires the detection amount related to the magnitude of the voltage V generated in the measurement target 9 from the voltage sensor 3.

In step S3, the measuring device 1 corrects the detection amount related to the magnitude of the current I detected by the current sensor 2 based on the sensor information 22 from the current sensor 2.

In the present embodiment, as described with reference to FIG. 5, the measuring device 1 acquires the approximation function indicating the relationship between the phase delay of the detection amount and the frequencies based on the phase characteristic data 223 included in the sensor information 22, and calculates the phase delay by substituting the values of the measured frequencies into the approximation function. The measuring device 1 corrects the delay of the time-series detection amount by the current sensor 2 based on the calculated phase delay.

In step S4, the measuring device 1 calculates the measurement amount of the electric power P transmitted to the measurement target 9 based on the corrected detection amount of the current sensor 2 and the detection amount of the voltage sensor 3.

As a concrete example, the measuring device 1 calculates the effective value Iᵣₘₛ of the current I based on the corrected detection amount of the current sensor 2, and calculates the effective value Vᵣₘₛ of the voltage V based on the detection amount of the voltage sensor 3. Further, the measuring device 1 acquires the phase difference θ between the corrected time-series detection amount of the current sensor 2 and the time-series detection amount of the voltage sensor 3.

Then, the measuring device 1 calculates the average value of the power P transmitted to the measurement target 9 by using the effective value Iᵣₘₛ of the current I, the effective value Vᵣₘₛ of the voltage V, and the phase difference θ of the current I with respect to the voltage V, as in the above formula (1).

In this manner, the measuring device 1 calculates the measurement amount of the measurement target 9 based on the corrected detection amount. Examples of the measurement amount of the measurement target 9 include the instantaneous values of the current I and the voltage V, the maximum values, the effective values, and the average values of the current I and the voltage V, and the instantaneous value and the average value of the power P.

When the process of step S4 is completed, a series of processes of the measurement method according to the present embodiment is terminated.

Hereinafter, operational effects of the present embodiment will be described in detail.

The measuring device 1 according to the present embodiment communicates with the current sensor 2 that holds the sensor information 22 including the information on the detection amount specific to the current sensor 2. The measuring device 1 includes the sensor information acquirer 20 that functions as an acquirer for acquiring the sensor information 22 from the current sensor 2 and the detection amount of the current I detected by the current sensor 2. The measuring device 1 includes the processor 100 functioning as a processor for correcting the detection amount acquired by the detection signal acquirer 30 based on the sensor information 22, and the measurement amount calculator 40 functioning as a calculator for calculating the measurement amount of the current I for the measurement target 9 based on the detection amount corrected by the processor 100.

Similarly, for the voltage sensor 3, the measuring device 1 corrects the detection amount of the voltage V detected by the voltage sensor 3 based on the sensor information 32 including the information on the detection amount specific to the voltage sensor 3, and calculates the measurement amount of the voltage V for the measurement target 9 based on the corrected detection amount.

The recording medium constituting the storage 50 in the present embodiment is a computer-readable recording medium in which a program to be executed by a computer constituting the measuring device 1 is recorded. The program recorded in the storage 50 includes an acquiring step (S2 and S3) of acquiring the sensor information 22 from the current sensor 2 and the detection amount detected by the current sensor 2. The program includes a processing step (S3) of correcting the detection amount acquired in the acquiring step (S2) based on the sensor information 22, and a calculating step (S4) of calculating the measurement amount of the measurement target 9 based on the detection amount corrected in the processing step (S3).

Similarly, for the voltage sensor 3, the program executes processing for calculating the measurement amount of the measurement target 9 using the detection amount of the voltage sensor 3 corrected based on the sensor information 32.

According to these configurations, when the measurement amount of the measurement target 9 is calculated, the detection amount specific to the current sensor 2 included in the sensor information 22 acquired from the current sensor 2 itself is used. In the first place, the measurement amount of the measurement target 9 is calculated based on the detection amount of the current sensor 2, and thus may be affected by the detection performance of the current sensor 2.

Therefore, by using the sensor information 22 when calculating the measurement amount, the detection error caused by the detection performance of the current sensor 2 can be considered. Therefore, the measurement amount in consideration of the detection performance of the current sensor 2 can be acquired. As a result, the measurement accuracy, which is the measure accuracy of the measurement amount acquired based on the detection amount of the current sensor 2, can be increased.

Further, according to these configurations, since the sensor information 22 is acquired from the current sensor 2 itself, even when the current sensor 2 connected to the measuring device 1 is replaced with another current sensor 2 that holds its own sensor information 22, the measurement accuracy can be similarly improved.

Further, in the present embodiment, the sensor information 22 includes the phase characteristic data 223 as information indicating the frequency characteristic of the phase related to the detection amount detected by the current sensor 2. Then, the processor 100 corrects the detection amount of the current sensor 2 based on the phase characteristic data 223, and the measurement amount calculator 40 calculates the measurement amount of the measurement target 9 based on the detection amount corrected by the processor 100. Examples of the measurement amount to be calculated include a waveform, an instantaneous value, an effective value, and an average value of the current I flowing through the measurement target 9, and a waveform, an instantaneous value, and an average value of the power P transmitted to the measurement target 9.

According to this configuration, since the detection amount of the current sensor 2 is corrected, a detection error caused by a phase delay of the detection amount in the high frequency band of the current sensor 2 can be suppressed. Therefore, the measure accuracy of the measurement amount of the measurement target 9 can be improved.

In the present embodiment, the sensor information 22 includes the amplitude characteristic data 224 as information indicating the frequency characteristic of the amplitude related to the detection amount detected by the current sensor 2. Then, the processor 100 corrects the detection amount of the current sensor 2 based on the amplitude characteristic data 224, and the measurement amount calculator 40 calculates the measurement amount of the measurement target 9 based on the detection amount corrected by the processor 100.

According to this configuration, since the detection amount of the current sensor 2 is corrected, a detection error caused by an amplitude attenuation of the detection amount in the high frequency band of the current sensor 2 can be suppressed. Therefore, the measure accuracy of the measurement amount of the measurement target 9 can be improved.

The measuring device 1 according to the present embodiment communicates with each of the current sensor 2 and the voltage sensor 3 as a plurality of sensors. Then, the sensor information acquirer 20 acquires the sensor information 22 from the current sensor 2 and acquires the sensor information 32 from the voltage sensor 3, and the processor 100 corrects the detection amount of the voltage sensor 3 based on the acquired sensor information 32. That is, the sensor information acquirer 20 acquires the sensor information for each sensor of the current sensor 2 and the voltage sensor 3, and the processor 100 corrects the detection amount based on the sensor information for each sensor.

Then, the measurement amount calculator 40 calculates the measurement amount of the measurement target 9 based on the plurality of detection amounts corrected by the processor 100 for each sensor of the current sensor 2 and the voltage sensor 3. For example, the measurement amount calculator 40 calculates the average value of the electric power P transmitted to the measurement target 9 as in the above formula (1) based on the detection amount of the current sensor 2 after correction and the detection amount of the voltage sensor 3 after correction by the processor 100.

According to this configuration, since a component of a detection error caused by the detection performance of the current sensor 2 and the voltage sensor 3 is reduced in the measurement amount calculated by the measurement amount calculator 40, the measurement accuracy of one measurement amount measured using a plurality of sensors can be increased.

In addition, since the measuring device 1 acquires the sensor information for each of the current sensor 2 and the voltage sensor 3, even in a case where the connection positions of the current sensor 2 and the voltage sensor 3 in the measuring device 1 are switched and measurement is performed, the detection amount of each sensor is appropriately corrected, and thus it is possible to similarly improve the measurement accuracy.

Although embodiments of the present invention have been described above, the embodiments merely illustrate part of application examples of the present invention, and no limitation of the technical scope of the present invention to the specific configurations of the embodiments described above is intended.

For example, although the sensor information is stored in the voltage sensor 3 in addition to the current sensor 2 in the present embodiment, the sensor information may be stored only in the current sensor 2 or only in the voltage sensor 3.

Further, in the present embodiment, the phase characteristic data 223 is used to correct the detection amount of the current sensor 2, but instead of this, the group delay characteristic data may be used to correct the phase.

In the present embodiment, the measurement target 9 is one electric line, but the measurement target 9 may be three electric lines that transmit three-phase AC power. Even in this case, three current sensors 2 and three voltage sensors 3 may be installed in the measurement target 9, and the measuring device 1 may calculate the three-phase AC power on the basis of the three pieces of sensor information 22 and the three pieces of sensor information 32 acquired from the respective sensors as in the present embodiment.

Further, in the present embodiment, the detection amounts related to the AC current and the AC voltage of the measurement target 9 are detected using the current sensor 2 and the voltage sensor 3, but the detection amounts related to the DC current and the DC voltage of the measurement target 9 may be detected using the current sensor 2 and the voltage sensor 3.

In the present embodiment, the measuring device 1 is provided with the operation interface 10 and the communicator 60. However, for example, at least one of the operation interface 10 and the communicator 60 may be omitted.

Further, in the present embodiment, both of the current sensor 2 and the voltage sensor 3 detect the detection amount in a non-contact manner with respect to the measurement target 9, but the sensor connected to the measuring device 1 may detect the detection amount in contact with the measurement target 9. For example, the sensor may be a high-voltage probe that brings a probe into contact with the measurement target 9, converts a high voltage of the measurement target 9 into a voltage that can be input to the measuring device 1, and outputs the converted voltage as a detection amount. Alternatively, the sensor may be a shunt that brings a probe into contact with the measurement target 9, converts a large current into a voltage that can be input to the measuring device 1, and outputs the converted voltage as a detection amount.

The present application claims priority based on JP 2021-203454 filed on Dec. 15, 2021, in Japan, the entire contents of which are incorporated by reference herein.

### REFERENCE SIGNS LIST

1 Measuring device
2 Current sensor (sensor)
3 Voltage sensor (sensor)
20 Sensor information acquirer (acquirer)
22, 32 Sensor information
30 Detection signal acquirer (acquirer)
40 Measurement amount calculator (calculator)
100 Processor (processor)

1. A measuring device for communicating with a plurality of sensors, the measuring device comprising:
an acquirer configured to acquire, from each of the sensors, information on a detection amount specific to the sensor and the detection amount detected by the sensor;
a processor configured to correct, for each of the sensors, the detection amount acquired by the acquirer based on the information; and
a calculator configured to calculate each of measurement amounts of a measurement target based on each of the detection amounts corrected by the processor, and each of the sensors is replaceable.

2. The measuring device according to claim 1, wherein
the information on the detection amount specific to the sensor includes information indicating a frequency characteristic of a phase of the detection amount, and
the processor is configured to correct the detection amount based on the information indicating the frequency characteristic of the phase.

3. The measuring device according to claim 1- or 2, wherein
the information on the detection amount specific to the sensor includes information indicating a frequency characteristic of an amplitude of the detection amount, and
the processor is configured to correct the detection amount based on the information indicating the frequency characteristic of the amplitude.

4. (canceled)

5. A computer-readable recording medium having recorded thereon a program for causing a computer that communicates with a plurality of sensors, each of which is replaceable, to execute steps of:
acquiring, from each of the sensors, information on a detection amount specific to the sensor and the detection amount detected by the sensor;
processing to correct each of the detection amounts acquired by the acquirer based on the information; and
calculating each of measurement amounts of a measurement target based on each of the detection amounts corrected by the processor.

6. A measuring device for communicating with a plurality of sensors, the measuring device comprising:
an acquirer configured to acquire, from each of the sensors, information on a detection amount specific to the sensor and the detection amount detected by the sensor;
a processor configured to correct, for each of the sensors, the detection amount acquired by the acquirer based on the information; and
a calculator configured to calculate each of measurement amounts of a measurement target based on each of the detection amounts corrected by the processor,
and one of the sensors is switchable to another one.

7. A measuring device for communicating with a plurality of sensors, the measuring device comprising:
an acquirer configured to acquire, from each of the sensors, information on a detection amount specific to the sensor and the detection amount detected by the sensor;
a processor configured to correct, for each of the sensors, the detection amount acquired by the acquirer based on the information; and
a calculator configured to calculate each of measurement amounts of a measurement target based on each of the detection amounts corrected by the processor,
and the sensors are connectable to the measuring device,
and the connection position of each of the sensors is switchable.
